(19) United States
(12) Patent Application Publication
(10) Pub. No.: US 2017/0219552 A1
(43) Pub. Date: Aug. 3, 2017

(54) Method and apparatus for determining the rate of sap-content variation in living plants, and relating that to soil water tension, and transmitting the collected information

(71) Applicants: Reinoud Jacob Hartman, Nanaimo (CA); Terry Lawrence St. Hilaire, Dryden (CA)

(72) Inventors: Reinoud Jacob Hartman, Nanaimo (CA); Terry Lawrence St. Hilaire, Dryden (CA)

(21) Appl. No.: 15/013,903

(22) Filed: Feb. 2, 2016

(51) Int. Cl. G01N 33/24; G01N 33/00; G01N 27/12

(52) U.S. Cl. CPC G01N 33/246; G01N 27/12; G01N 33/0098

(57) ABSTRACT

Methods and apparatuses are described for the measurement of sap content variation using one or more metallic probes inserted through the external surface into the underlying sap-carrying layers of the plant, connected to a Device using a Micro-controller and an algorithm to create and report an electrical signal, and, and in another embodiment measuring and relating sap content to soil water tension using such Device, that reads such multiple sensor types, and in another embodiment configuring the Device to transport such sensor and relative information via wide area or wireless methods.

### FIELD OF THE INVENTION

This invention is related to the field of electronic sensing for precision agriculture, agronomy, landscape management, greenhouse management, and forestry, and more particularly for purposes of monitoring sap content in trees and plants as it is related to irrigation practice, and to the study of plant physiology, and more specifically in providing tools and information to aid modifying traits in plants, managing their growing environments and optimizing water use in irrigation and managing fertilizer and nutrient uptake and by inferral managing the level of moisture in soils or other media and for purposes of establishing precise control over those parameters according to user preference. Other uses can be found in providing measurements for Water Balance determination, Plant Transpiration, Disease Effects, Greenhouse Management, Irrigation Scheduling, and Phytoremediation.

### BACKGROUND

Plants feed the world and provide critical resources to mankind. Increasing population-growth-driven demands on existing global food, resource and water supplies, combined with the often-negative effects of climate change on existing plant growing patterns, demand that more efficient and sustainable processes for converting the earth's resources into human sustenance be sought. Soil and water are key resources which are being stressed by erosion, poor management practices and over-use. Improving our understanding of the needs of plants and their relationship with soil and water, and providing tools to help plant science researchers reduce negative impacts of human resource utilization, and help growers minimize their use of natural resources is becoming essential.

### PRIOR ART

Current techniques and designs aimed at automatic plant irrigation focus principally on concepts involving timing and hard-wired electrical and mechanical (through piping) designs—see (1,2) below. Such concepts incorporate simple concepts for irrigation, typically based on timing schedules and perhaps incorporating local weather information. They are also not easily scalable due to the direct (area squared) relationship between irrigation area monitored and its subsequent equipment coverage needs.

Ref (3) by Gensler describes a method and apparatus for determining plant water content by inserting two metallic, shaped sensor electrodes in the plant, one through the petiole and the other into the root structure. The technique takes into account the difference in petiole diameters by noting the total area of the surface of the sensor within the plant. As water content is increased within the plant, this becomes manifest in a greater proportion of the surface area of the sensor inside the plant that is wetted. A capacitance measurement between the two sensor electrodes is then made at the interface between the surface and the water in the plant. Plant water content is derived from the ratio of measured capacitance to measured sensor surface area within the plant. This technique then is based on capacitance measurements between a number of sensors that are required to be placed in the plant itself together with highly specific and manual measurements at a given plant and is thus highly labor intensive.

By contrast, Ref (4) describes a more conventional technique for deriving sap flow based on heating the plant stem. It requires that two holes be made in the plant stem, one above the other, and the insertion of two temperature monitoring probes (one of which is a heating probe and the other is not). Current is then supplied to the heating probe and the temperature difference between the two probes established. Comparison of the temperatures thus allows a flow index to be calculated which can be related to sap flow within the stem.

Ref (5) describes a technique based on plant stomatal characteristics and, through measuring the diffusive resistance of plant stomata, then determining water loss by plants. The measurement technique concerns the determination of diffusive resistance through the measurement of water vapor loss through a single leaf—such water loss increases the air humidity in the container holding the leaf. The diffusive resistance is then related to the rate of increase of humidity in the container.

This invention differs from all prior art contained in Refs. (3, 4, 5) in both its concept and its execution. It is simple with only two wire electrodes and a local microcontroller for introducing an active signal (a pulse with specific duty cycle) into a plant stem and then signal processing algorithms local to the plant for deriving an output representative of the relative rate of sap content variation in real-time within a living plant. Such data can then be made available to remote and local users, as required. This invention is also highly scalable to very large areas (10-100s acres). More specifically, and with reference to the above Prior Art, this invention differs as follows:
Refs (1, 2): these are physical irrigation system designs whereas this invention is focused on a new technique for determining the rate of sap-content variation in living plants, and relating that to soil water tension, and transmitting the collected information. It is based on determining sap-content variation in a plant(s) together with local and/or remote data transmission and analysis of such data to enable growers (e.g.) to understand the soil characteristics in a defined area for possible irrigation schemes that would use such system designs as mentioned in Refs (1,2).
Ref. (3): this technique concerns the derivation of capacitance between a number of sensors that are required to be placed in the plant itself (and its root structure) together with highly specific and manual measurements at a given plant and is highly labor intensive. It requires detailed on-site measurements of the implanted sensors and that area which has been wetted by the plant. By contrast, this invention is an electrical one based on pulsed electrical signals transmitted to two simple sensor pads together with a localized microcontroller with embedded signal processing for deriving plant resistance. Additionally, this different plant parameter data can then be stored for either local data review or transmission over other wide area methods of communication including but not limited to infrared, Ethernet and serial connectivity.
Ref. (4) describes a more conventional technique for deriving sap flow based on heating the plant stem, requiring sufficient voltage/current equipment in order to generate the required heating effect within the plant. This invention does not derive sap flow based on heating of the plant—rather, it is based on local electrical (voltage) signal measurements together with a localized microcontroller with embedded processing for deriving plant resistance. This invention relies on very low electrical power requirements based on battery-less and solar charging technologies within a very small physical footprint.
Ref. (5) describes a single leaf-based technique to measuring the diffusive resistance of plant stomata and then relating that single measurement to plant water loss. This is highly manual technique conducted in a laboratory-type environment and associated mains supplied capital equipment. By contrast, this invention is a highly scalable, field-based technique using the plant itself as the sensor of the local water environment. This invention relies on very low electrical power requirements based on battery-less and solar charging technologies within a very small physical footprint. This invention also has many extra embodiments including the incorporation of soil tension sensors (e.g.), enabling the recorded trend of the plant sap content to be reconciled with irrigation methods.

### REFERENCES

1. CA 2544528 Method and system for controlling irrigation using computed evapotranspiration values by Marian, M (2004).
2. CA 2735998 Method and device for the automatic regulation of plant irrigation by Schmidt. W, Toprak, Y (2009).
3. U.S. Pat. No. 6,870,376 Method and apparatus for determining plant water content by Gensler, W G (2005).
4. U.S. Pat. No. 4,745,805 Process and device for the measurement of the flow of raw sap in the stem of a plant such as a tree by Granier, A F (1988).
5. U.S. Pat. No. 4,160,374 Apparatus for measuring the diffusive resistance of plant stomata by Crump, T J, Crump, J M (1979)

### SUMMARY OF THE INVENTION

This invention is a method and apparatus for measuring the relative rate of sap content variation in real-time within a living plant. It may additionally relate that to other local environmental variables and may additionally communicate the information using wide area means.

It comprises one or more stainless steel probes inserted into the area of a plant. Each probe is connected by wire to a near-by controller which contains an algorithm that causes the controller to transmit and receive electrical pulses via the inserted probe(s). The characteristics of the read or returned pulses are then internally processed to derive a measurement of the availability of ions carried in sap within the measurement area. This measurement is claimed to be directly related to sap content in the area of the plant the user has selected for investigation. This method differs from conventional sap-flow measurement devices which are typically based on introducing high power-demand thermal effects into the plant.

A second embodiment incorporates soil tension sensors, enabling the recorded trend of the plant sap content to be reconciled with irrigation methods.

A third embodiment integrates an RF transmission module into the device enabling real-time reporting in conventional in-field applications.

A fourth embodiment integrates other wide area methods of communication including but not limited to infrared, Ethernet and serial connectivity.

The collected sensor information is stored electronically in memory elements of the micro-controller, and is made available for user-initiated download, automated download, or is sent on via the RF transmission module to another device or, via a gateway device, via the Internet to a web portal that allows an end-user of other machine to access the sensor information and or any relational or interpreted data created in the micro-controller.

### Advantages

The object of the invention is to bring the ability to track sap content variation in plants to common growers through simplification by: reducing hardware requirements; installation complexity; and, plant rejection of sensors compared to sap-flow measurement methods currently employed. This information will lead to reduced user resource demands, increased growing efficiency, and increased output per land unit while reducing resource consumption.

Instead of the conventional techniques of using sap measurements to gauge irrigation needs such as introducing a thermal gradient in the sap that can be tracked, or manual methods using pressure to extract available sap from a leaf, this method of using electronic pulses to track ions in plant sap, results in a far lower-cost and longer lasting installation, and while very useful to scientists and plant researchers, will allow growers of all types, including production-oriented farmers and land managers, access to important information to guide irrigation practice.

Introducing thermal effects in medium to large-sized plants as is commonly done to measure sap content demands large amounts of energy relative to levels at which micro-processors conventionally operate. This invention's method of using harvested energy is ideal for in-field applications as it reduces costs and complexity for users due to the devices being more autonomous, lower cost and demanding less maintenance. Also, technologies which enable RF communication in large area applications using energy harvesting in battery-less devices have already been commercially deployed and so it would be ideal if those same devices measure plant sap; however, such devices have insufficient thermal energy capacity needed for conventional sap flow measurements. The invention is based on a measurement process that is not based on thermal effects and thus it allows such low energy-demanding devices to measure sap content.

Knowing sap-content rates allows researchers to better understand the physiology of a living and functioning plant, enabling better understanding of turgor, stress and vigor that is useful in studies of the processes that affect the growth of plants. For farmers, foresters and growers of all types, knowing sap content in relation to environmental inputs can aid in determining irrigation requirements, the effect of fertilizers and pesticides, impacts of climate and a broad range of inputs that enhance or detract from the usefulness of plants to man.

Having sap content information readily at hand and enabling its integration into control processes automatically, means that baseline sap content thresholds for various stages of development for each plant type in question can be utilized to provide automated warnings to growers and land managers related to the application of irrigation, or the need for ventilation or shading for example.

### DESCRIPTION OF THE INVENTION

Attached FIG. 1 shows a typical installation of the three embodiments of the invention; more specifically:
a. The first embodiment shows two sap-content sensor probes 1 inserted into the branch of a plant. Wires from the sensors 2 are routed back to a Micro-controller 3 contained within a Device 4 and are connected at input/measurement terminals 10.
b. As a second embodiment, a soil moisture or soil moisture tension sensor 5 may also be connected to the Micro-controller.
c. As a third embodiment, an RF device and antenna 6 may also be connected to the Micro-controller emitting radio waves 9 used to communicate the sensor and relational data.
d. Optionally, 7 shows a solar panel used to harvest energy to operate the Micro-controller.
e. As a fourth embodiment, such measured and processed data can be integrated into other wide area methods of communication including but not limited to infrared, Ethernet and serial connectivity.

In Window A, it can be seen that sap-content sensor probes 1 penetrate the outer layers of the plant (shown in both Plan and Section views) and extend into the moist underlying layers 8.

Some key points to note include:
a. Sap content sensors 1 may be either staple-, or probe-, shaped.
b. Sensor system deployment is very simple.
c. Measured data can be logged or transmitted.
d. One or more plants can be monitored from one Device.

The invention focusses on measuring the plant's resistance to an externally-originating process electrically. Once the system has been deployed in/around the plant, measurements in that Measurement Area are:
a. Immediately after the sensor pads 1 have been shorted, a snapshot of the voltage is taken after a current of less than 50 uA has been applied for 100 uS duration, where this duty cycle is quoted as representative and an example only.
b. The underlying change in resistance in the plant thus modifies the voltage at an exact point in time.
c. The characteristics of the read or returned pulses are then internally processed in the Micro-controller 3 to derive a measurement of the availability of ions carried in sap within the Measurement Area.
d. Such measurements are made in real-time.

### DISCLOSURE OF THE INVENTION

(a) Has the Invention been disclosed outside of your company (e.g., disclosed in white paper posted on company website) or commercialized (e.g., a press release announcing availability for sale of a product including the invention) in any way?

No.
(b) Invention Dates

Invention conceived on: 20 Jun. 2014

Location where invention was conceived:

The invention was conceived in Nanaimo, BC and in Dryden, Ontario during a communication between the inventors.

Who was there when the invention was conceived and sorted out?
Reinoud J Hartman
Terry St. Hilaire

### DRAWINGS

FIG. 1 shows a typical installation of the three embodiments of the invention.

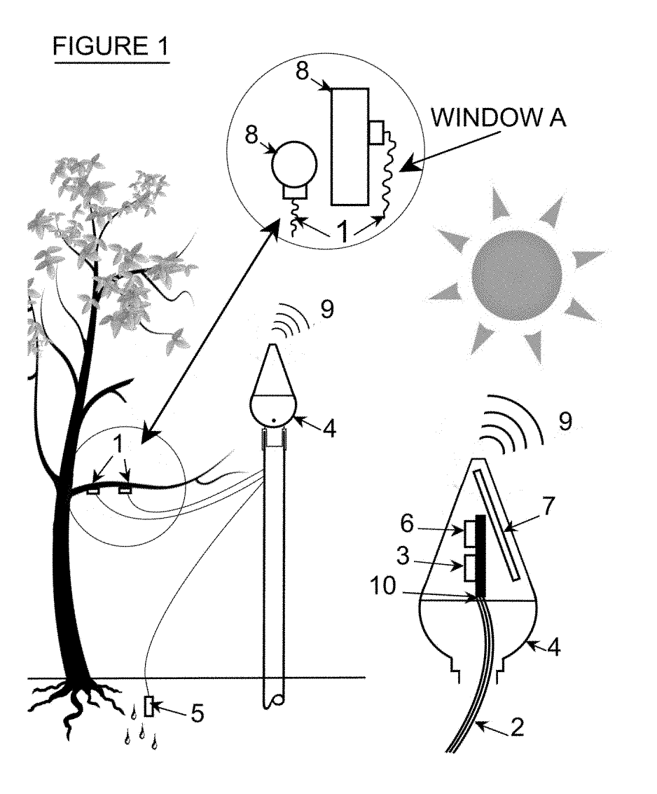

1. A sensor comprising one or more metallic sharp-tipped probes each connected electrically by a conducting wire to input and measurement terminals of a Micro-controller, which Micro-controller in a second embodiment may also be connected electrically by a conducting wire to a soil water tension sensor and which Micro-controller in a third embodiment may also have electronically integrated an RF module, which Micro-controller is incorporated in a Device. Said Sensors operated using the Micro-controller of claim 1 operating at very low levels of external, battery or harvested energy, said Micro-controller creating and reading current-level electrical signals of 50 uA or less;

2. Said sharp-tipped sensor(s) of claim 1 inserted through the outer surface of a plant or tree towards the interior of its trunk, or a branch, or stem, or leaf in a Measurement Area so as to penetrate or pass into one or more of the moist sub-surficial layers such that electrical signals can be introduced into those plant layers via the sensors, and such signals being of such low power as to induce both no meaningful thermal effect in, or interfere with the functioning of, the plant or any area of the plant;

3. The micro-controller of claim 1 containing an Algorithm which causes the Micro-controller to generate and drive via the Sensors electrical pulses of a specified duration at a specified time interval and interval between pulses, and for a specific or variable number of repetitions, at a specific and variable intensity or magnitude, all without inducing any significant thermal effect, and then perceiving and reading the residual effect of one or all of the electrical pulses;

4. The Algorithm of claim 3 which relates the momentary voltage measurement of the read residual pulses relative to the transmitted pulses, to the available ions in the fluid within the moist sub-surface plant layers, and which may additionally relate them to the readings also so derived from the soil tension sensors;

5. The Algorithm of claim 3 which provides a numerically-expressed result directly related to the momentary proportional quantity of sap ions in the Measurement area relative to the preceding reading, and may additionally relate the numerically-expressed result to measured soil water tension;

6. The Numeric Readings of claim 5, correlating the density of available ions in direct proportion to the variable amount of sap in the measurement area at a moment in time, communicated to a user or machine interface in digital or electronic form;

7. The Device of claim 1 configured with an RF module which transmits the sensor readings to a gateway device connected to the internet, of which transmits the sensor readings to a data logger for later download.
